(19) United States
(12) Patent Application Publication
(10) Pub. No.: US 2026/0018316 A1
(43) Pub. Date: Jan. 15, 2026

(54) SOFT AND DRY ELECTRODE

(71) Applicant: Dätwyler Schweiz AG, Schattdorf (CH)

(72) Inventors: Samira JAFARI, Zurich (CH); Tom LAMBRECHTS, Diest (BE); Camille Joël GILBERT, Amsteg (CH)

(73) Assignee: Dätwyler Schweiz AG, Schattdorf (CH)

(21) Appl. No.: 19/114,690

(22) PCT Filed: Sep. 7, 2023
(86) PCT No.: PCT/CH2023/050031

§ 371 Date: Mar. 24, 2025

(30) Foreign Application Priority Data
Sep. 26, 2022 (CH) 001112/2022

(51) Int. Cl. H01B 1/12; A61B 5/28; A61B 5/291; A61B 5/296; H01B 5/00

(52) U.S. Cl. CPC H01B 1/124; A61B 5/28; A61B 5/291; A61B 5/296; H01B 5/00

(57) ABSTRACT

A soft electrode for measuring bioelectric signals is made of elastomeric material, has conductive properties, and includes a central connector portion with an attachment element for attaching the electrode to a measuring apparatus, and flexible contact members. The electrode has a connector side on the side of the attachment element, and a contact side opposite the connector side. The contact members are arranged around the central axis of the electrode connected with the connector portion and protrude on the contact side for contacting a measurement area of an individual. Each contact member has a flexible leg and a foot. The leg has a longitudinal cavity formed by an inner wall curved around a longitudinal axis of the contact member. The longitudinal cavity remains open on the connector side and is closed on the contact side by the foot. The foot and the leg form a continuous radially inner contact surface.

### TECHNICAL FIELD

The invention relates to soft and dry electrodes for detection of bioelectric signals in applications such as electroencephalography (EEG), electrocardiogramhy (ECG) or electromyography (EMG).

### TECHNICAL BACKGROUND

Commercially available ‘dry’ EEG headsets are often equipped with metal dry electrodes, which causes subjects to feel pain after wearing the headsets for a while. A possible solution is combining such electrodes with a spring-like system to avoid high skin pressure.

Yet another approach is the use of soft polymer-based dry electrodes. By mixing the elastic polymer with additives the conductivity can be improved while maintaining the required elasticity for high user comfort. The polymer based dry electrodes can have a comb shape design (fingers or legs) to improve skin contact on hairy skin (e.g. on the scalp). Such fingers or legs can have at least a partial coating on a surface of the electrode in contact with the skin in order to lower the skin impedance and provide an improved signal quality.

Thus, soft and dry electrodes are increasingly used for long term biopotential measurements such as EEG and ECG. Next to being soft, the additional fact that such electrodes can be applied without the use of a conductive gel provides the measurement procedure with considerable benefits such as a decreased risk of skin irritation and the avoidance of a decrease of signal quality due to gel drying.

Examples of such soft and dry electrodes are described by Chen et al. in “Polymer-based dry electrodes for high user comfort ECG/EEG measurements” (Chen, Yun-Hsuan; Op de Beeck, Maaike; Carrette, Evelien; Vanderheyden, Luc; Grundlehner, Bernard; Mihajlovic, Vojkan; Boon, Paul; Van Hoof, Chris; Apprimus Verlag; Aachen; 8th International Conference Exhibition on Integration Issues of Miniaturized Systems-MEMS, NEMS, ICs and Electronic Components: 2014; pp. 329-336), Chen et al. in “Soft, Comfortable Polymer Dry Electrodes for High Quality ECG and EEG Recording” (Sensors 2014, 14, 23758-23780; doi: 10.3390/s141223758) or WO2016080804.

These soft and dry electrodes comprise a base plate and a plurality of pins for contacting an area of interest to be measured. The pins may have a tapered portion and a protruding portion. The electrode tips are made of a flexible or soft matrix material which is provided with an electrically conductive material. The electrodes may have a knob on its upper side of the base plate opposite of the pins for electrically connecting the electrode.

Upon exertion of force on the soft electrode (e.g. by means of a strap, band, headset or head-cap) the legs may move in an uncontrolled manner, not offering the intended brush function to move aside hair and provide for a direct contact between electrode and skin surface.

One possible solution for this problem is a pre-orientation of the legs (as described in EP2827770) so that on applying the electrode to the subject area (e.g. a scalp) these legs are disposed at a non-perpendicular angle to that subject area. A downside of this approach however is the adopted manufacturing process involving a 3D-printing step which is not suitable for up-scaling towards high volume productions.

JP2019097733relates to an electrode for measuring brain activity. The electrode has a stiff support body and several arms attached at the side of the stiff support body. A ball is formed at the tip of the arms to contact the scalp of a person. The arms are flexible and bend when a force is applied to the electrode. The electrode has a complex shape with several undercuts, which makes it difficult to manufacture in a cost-efficient way.

WO2022047595 describes a soft and dry electrode with a flexible support body and a plurality of pins parallel to a central axis of the support body. Upon force exertion on the top surface of the support body, the flexible support body starts bending such that the tip of the pins or legs move in an outward direction (i.e. away from the centre of the electrode) in order to ‘brush aside’ any hair that is hindering direct contact between electrode and bare skin surface of the individual to be measured.

### SUMMARY OF THE INVENTION

It is an objective of the invention to provide a soft and dry electrode for measuring bioelectric signals of an individual avoiding the problems of the prior art and being suitable for high volume production. It is a further objective of the invention to improve the form of a soft and dry electrode to increase brushing through the hair in order to optimize contact between the tips of the pins and the scalp/body of the individual.

At least one of the objectives of the present invention is achieved by a soft and dry electrode according to claim 1. The soft and dry electrode for measuring bioelectric signals of an individual is made of elastomeric material and has conductive properties. The electrode comprises a central connector portion with attachment means for attaching the electrode to a measuring apparatus and a plurality of flexible contact members. The electrode has a connector side on the side of the attachment means, and a contact side opposite the connector side and facing the individual when applying the electrode to the individual. The connector portion further defines a central axis of the electrode arranged centrally through the contact side and the connector side of the electrode. The plurality of contact members are arranged around the central axis and connected with the connector portion. The plurality of contact members further protrudes on the contact side for contacting an area of interest of the individual for measurement. Each contact member of the plurality of contact members comprises (or may consist of) a flexible leg portion and a foot portion at a free end of the flexible leg portion. The flexible leg portion has a longitudinal cavity formed by an inner wall curved around a longitudinal axis of the contact member, wherein the longitudinal cavity remains open on the connector side and is closed on the contact side by the foot portion. The foot portion and the flexible leg portion form a continuous radially inner contact surface.

The electrode thus has a structure where the inner wall of the flexible leg portion can translate smoothly into the foot portion. Manufacturing of the electrode does not require complex tooling e.g. due to undercuts. At the same time a high flexibility of the leg portion is maintained due to the “hollow” structure, i.e. the longitudinal cavity, of the curved wall, which weakens the leg portion. This allows the contact member to bend when the electrode is placed with its contact side on an individual and force is applied on the connector side. Due to the weakening of the leg portion on the radially outer side, the bending occurs in a way that the foot portion moves in a radial outward direction along the skin of the individual and may brush through hair. The continuous radially inner contact surface thus has no edges, steps or sudden changes in curvature, such that when applying force on the connector side of the electrode the contact member can smoothly move along the skin of the individual even when the actual contact zone moves along the contact surface. In addition, the outward movement of the foot portion when applying pressure on the connector side may be further enhanced by a tilt angle between at least a portion of the radially inner contact surface of the contact member and the central axis of the electrode. The contact members may thereby open towards the tip portion such that they move radially outwards upon contacting the area of interest of the individual.

The foot portion may be more rigid (or less flexible) in comparison with the flexible leg portion, but still maintains an elasticity due the elastomeric material of the electrode. This may be achieved by a solid design of the foot portion without a cavity or “hollow” structure. The bending of the contact member occurs along the leg portion.

In the context of the present invention “upper” refers to the connector side and “lower” refers to the contact side of the electrode. “Radial” is used in the context of the central axis of the electrode. The longitudinal axis of the contact member leads approximately through a centre of the foot portion and longitudinally along or through the leg portion, or in other words is a longitudinal axis along the longitudinal cavity. The tilt angle of the contact surface may be determined along the radially innermost line of the curved inner wall. The contact surface may be the surface of the inner wall facing radially inwards and which may contact the individual when the contact elements are bend due to forces applied on the connector side.

Further embodiments of the invention are set forth in the dependent claims.

In some embodiments the electrode may be made of elastomeric material, which is provided with conductive additives and/or is at least partially coated with a conductive coating.

In some embodiments the radially inner contact surface may be tilted, and a tilt angle between the radially inner contact surface and the central axis of the electrode is approx. 10 to 45 degrees and opens on the contact side.

In some embodiments good results have been achieved with a tilt angle between the radially inner contact surface and the central axis of the electrode is approx. 15 to 25 degrees, preferably about 17.5 to 20 degrees.

In some embodiments the tilt angle of the contact surface starting at a lower end of the leg portion to the lower end of the foot portion may continuously increase, preferably increase to about 35 to 45 degrees, more preferably to about 20 to 35 degrees. A tilt angle continuously increasing towards the tip of the contact element favours the sliding of the foot portion along the skin of an individual when a force is applied, and the leg portion starts to bend. The actual contact area of the contact element may thereby move from the very tip of the foot portion towards the inner side of the foot portion and lower inner side of the leg portion depending on the degree of bending of the contact element.

In some embodiments the contact surface only at the foot portion has an increased tilt angle and at the at least upper part of the contact members is approx. parallel to the central axis i.e. a tilt angle of approx. zero or close to zero degrees.

In some embodiments the thickness of the curved inner wall may decrease in a radially outward direction. Thereby, the radially outer part of the inner wall may distort more easily upon exertion of a force on the connector side improving the bending behaviour of the leg portion.

In some embodiments the foot portion may extend in radial outward direction over the inner wall of the leg portion.

In some embodiments the leg portion may further comprise a curved outer wall forming together with the curved inner wall the longitudinal cavity of the leg portion with an opening towards the connector side of the electrode. Thereby the longitudinal cavity of the leg portion is formed by a circumferential wall. The thickness of the curved outer wall may be smaller than the thickness of the curved inner wall.

In some embodiments an outer surface of the inner wall and/or an outer surface of the outer wall translates into the outer surface of the foot portion to form a continuous outer surface of the contact element. In other words, the entire contact element, i.e. leg portion and foot portion forms a smooth outer surface. In the context of the invention the outer surface is the surface of the respective portions facing away from the longitudinal axis thereby forming the convex side of the inner or outer wall of the leg portion.

In some embodiments each leg portion may be connected to adjacent leg portions at an upper end of the leg portion. The stability of the electrode at its connector side is thereby increased.

In some embodiments the contact member is tapered towards the foot portion such that the size of the cross-section continuously decreases.

In some embodiments the upper surface of the contact members may form together with the connector portion a dome-shaped upper surface.

In some embodiments a circumferential step may be formed between the connector portion and the plurality of contact members. Such a step enhances the bending behaviour of the electrode and reduces the required forces.

In some embodiments the plurality of contact members form a dome-shaped upper wall. The plurality of contact members may also form a flat upper wall perpendicular to the central axis.

In some embodiments the attachment means may be a knob formed by the connector portion of the electrode or a rigid connecting insert embedded in the connector portion of the electrode.

In some embodiments the longitudinal cavity has a semi-oval, semi-elliptic, oval or elliptic cross section.

In some embodiments the longitudinal cavity may be open or closed in radially outer direction.

In some embodiments the electrodes may comprise six to twelve contact members, preferably eight contact members, spaced around the central axis.

In some embodiments the plurality of contact members may be regularly arranged around the central axis of the electrode.

In some embodiments the connector portion and the plurality of contact members of the electrode may be integrally formed as single pieces of elastomeric material, with or without a rigid insert in the connector portion.

In some embodiments the electrode may have a diameter (around the central axis A) of 8 to 25 mm and a length (along the central axis) of 6 to 25 mm.

In some embodiments the contact members may have a length of 4 to 20 mm and an average cross-section in radial and/or tangential direction of 1.5 to 3.5 mm.

### BRIEF EXPLANATION OF THE FIGURES

The invention is described in greater detail below with reference to embodiments that are illustrated in the figures. The figures show:

FIG. 1 a first embodiment of a soft electrode under (a) a perspective view on the connector side, (b) a perspective view on the contact side, (c) a top view on the connector side, (d) a side view, (e) a cross-section along C1-C1 as shown in FIG. 1(c), (f) a cross-section along C2-C2 as shown in FIG. 1(c);

FIG. 2 a second embodiment of a soft electrode under (a) a perspective view on the connector side, (b) a perspective view on the contact side, (c) a top view on the connector side, (d) a side view, (e) a cross-section along C1-C1 as shown in FIG. 2(c), (f) a cross-section along C2-C2 as shown in FIG. 2(c);

FIG. 3 a third embodiment of a soft electrode under (a) a perspective view on the connector side, (b) a perspective view on the contact side, (c) a top view on the connector side, (d) a side view, (e) a cross-section along C1-C1 as shown in FIG. 3(c), (f) a cross-section along C2-C2 as shown in FIG. 3(c).

### EMBODIMENTS OF THE INVENTION

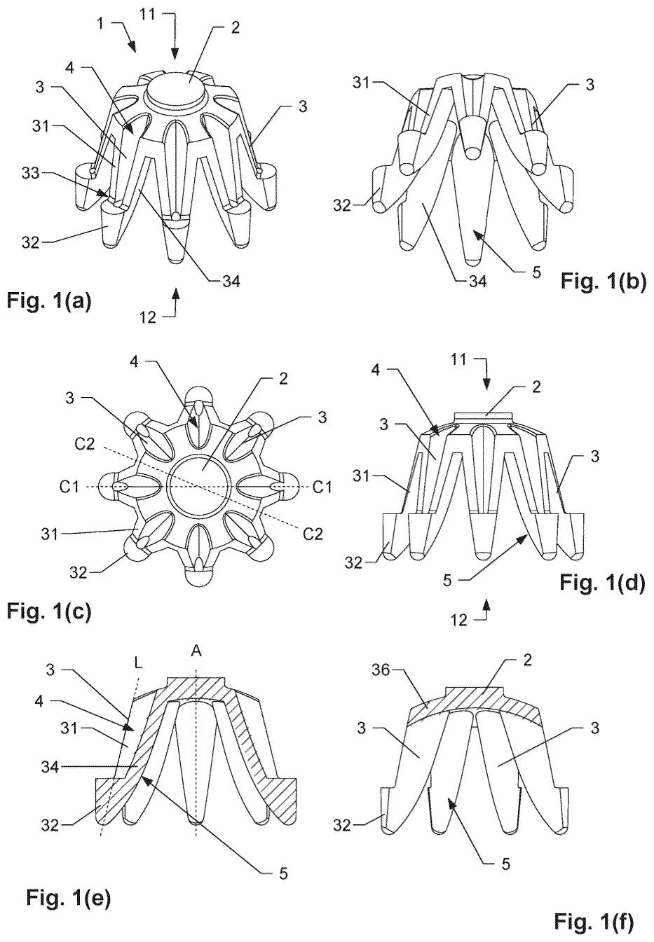

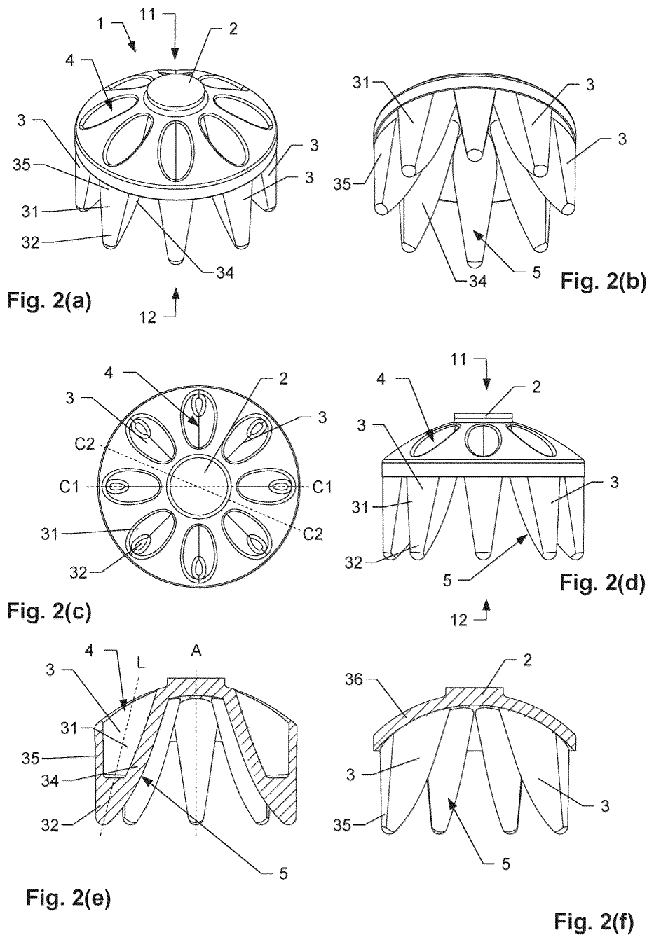

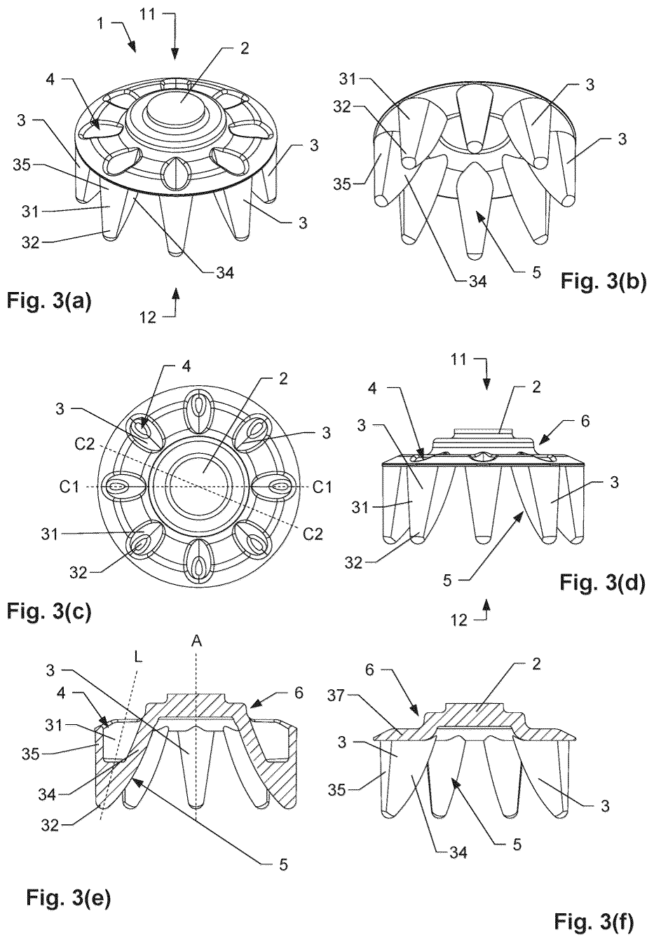

FIG. 1, FIG. 2 and FIG. 3 show three embodiments of a soft electrode 1 for measuring bioelectric signals of an individual. The electrodes 1 are made of elastomeric material to obtain a soft and flexible electrode and have conductive properties to be able to measure even small electrical signal on the skin of the individual. The electrodes 1 comprise a central connector portion 2 and a plurality of flexible contact members 3. The central connector 2 is provided with attachment means (not shown), e.g. in the form of a integral knob or an embedded insert with e.g. a knob, for attaching the electrode to a measuring apparatus, e.g. a head-set. Such attachment means are known in the art.

In the embodiments shown, the electrodes have eight contact members regularly spaced around the central axis.

The electrodes 1 further define a connector side 11 on the side of the attachment means and a contact side 12 opposite the connector side 11 and facing the individual when the electrode 1 is correctly applied, i.e. the connector side 11 is facing away from the individual. Thus, the electrodes are placed with the contact side facing the individual. The apparatus or head-set exerts a force onto the connector side 11 of the electrode 1 pressing the electrodes 1 with the contact side 12 onto the individual.

The central connector portion 2 defines a central axis A of the electrode 1, which runs through the connector and contact side 11, 12. The plurality of contact members 3 are regularly arranged around the central axis A and connected with the connector portion 2. The connector members 3 protrude from the connector portion 2 on the contact side 12 and contact a contact area of interest of the individual when the electrodes 1 are correctly applied. The connector side 11 with the attachment means faces away from the contact area of the individual.

Each of the contact members 3 of the plurality of contact members 3 comprises a flexible leg portion 31 and a more rigid foot portion 32 at the free end 33 of the flexible leg portion 31. The foot portion 32 is rigid in comparison with the flexible leg portion 31, but still maintains an elasticity due the elastomeric material of the electrode 1. The flexible leg portion comprises a longitudinal cavity 4 such that the flexible leg portion 31 may more easily bend when pressure is exerted onto the connector side 11 of the electrode 1. A longitudinal axis L is defined by the protruding contact members 3 and the longitudinal cavity 4 of the leg portion 3. The longitudinal cavity 4 is formed by at least a curved inner wall 34 curved around the longitudinal axis L of the contact member 3.

An outer surface of the inner wall 4, i.e. the surface facing the central axis A forms a continuous surface with the outer surface of the foot portion 32. In other words, the outer surface of the leg portion 31 smoothly translates into the outer surface of the foot portion 32. The innermost outer surface along the contact member forms a continuous radially inner contact surface 5. Different regions of the contact surface 5 may contact the individual depending on the degree of bending of the contact member 3 when pressure is applied onto the connector side 11.

The radially inner contact surface 5 is tilted with respect to the central axis A of the electrode 1. A tilt angle α between the radially inner contact surface 5 and the central axis A is approx. 15 to 25 degrees. In the embodiments shown in FIGS. 1-3 the tilt angle α is about 18 degrees. The tilt angle α opens towards the contact side 12, such that the foot portion 32 slides in a radially outward direction when pressure is applied onto the connector side 11 and the electrode is pressed with the contact side 12 onto the individual. Thereby, the more rigid foot portion 32 may slide along the skin/scalp of the individual and brush through hair (if present). At the same time the actual contact area on the contact member 3 increases and/or moves upwards along the contact member 3, i.e. towards the connector side 11.

To gain stability in the region of the connector portion 2 an upper end of the inner wall 34 of the leg portion 31 may be connected with an upper end of the inner wall 34 of the leg portion 31 of an adjacent leg portion 31.

The longitudinal cavity 4 remains open on the connector side 11 but is closed on the contact side 12 by the foot portion 32. The contact members 3 are tapered towards the foot portion 32 such that the size of the cross-section continuously decreases. The design of the electrode has no undercut and therefore allows the manufacturing with rather simple moulds using only two mould halves.

In the embodiments shown in FIG. 1-3 the tilt angle at the upper end of the leg portion 31 until approx. to the middle of the leg portion 31 is constant. It then continuously increases towards the lower end of the foot portion 32 to about 45 degrees. The foot portion 32 is efficiently guided in radially outward direction when force is applied onto the connector side 11 and the leg portion 31 starts to bend outwards.

The thickness of the curved inner wall 34 may decrease towards the outer side of the electrode. The curved inner wall 34 is thus less flexible on the inside than on the outside further promoting a guided outwards movement of the foot portion 32.

In the embodiment of FIG. 1 the longitudinal cavity 4 of the contact member 3 is open on the radially outer side. The leg portion 31 is formed by the inner wall 34 only. The foot portion 32 extends in radial outward direction over the inner wall 34 of the leg portion 31. The inner wall has a U-shaped cross-section and opens in radial outward direction. The cavity 4 has a semi-oval or semi-elliptic cross-section. The foot portion 32 has an oval or elliptic cross section.

In the embodiments of FIG. 1 and FIG. 2, the plurality of contact members 3 may form a dome-shaped upper wall.

In the embodiments of FIG. 2 and FIG. 3—as compared to the embodiment of FIG. 1—the longitudinal cavity 4 of the contact member 3 is closed on the radially outer side by a curved outer wall 35 curved around the longitudinal axis L. The curved inner wall 34 and the curved outer wall 35 form together the longitudinal cavity 4 of the leg portion 31 with an opening towards the connector side 11. The contact side 12 of the longitudinal cavity 4 is closed by the foot portion 32. The outer surface of the inner and outer wall 34, 35 translates smoothly into the outer surface of the foot portion 32. The outer surfaces of the leg portion 31 and the foot portion 32 form a continuous surface. The longitudinal cavity 4, the leg portion 31 and the foot portion 32 have an oval or elliptic cross-section.

The thickness of the outer wall 35 may be smaller than the thickness of the inner wall 34 to obtain higher flexibility on the radially outer side of the leg portion 31 than the radially inner side of the leg portion 31. Guiding of the foot portion 32 in a radially outwards direction when pressure is applied, is improved.

In comparison to the embodiment of FIG. 2, the electrode of the embodiment of FIG. 3 has a circumferential step 6 between the connector portion 2 and the plurality of contact members 3. In the shown embodiment the contact members form a flat upper wall (perpendicular to the central axis A). At the circumferential step 6 the upper wall 37 may bend slightly upwards when pressure is applied onto the connector portion 2. Embodiments with dome-shaped upper wall may also have a step between the connector portion 2 and the plurality of contact members 3

### REFERENCE SIGNS

1 electrode
11 connector side of electrode/connector portion
12 contact side of electrode
2 connector portion
3 contact member
31 flexible leg portion
32 rigid foot portion
33 free end/lower end
34 curved inner wall
35 curved outer wall
36 dome-shaped upper wall
37 flat upper wall
4 longitudinal cavity
5 inner contact surface
6 circumferential step
A central axis of electrode
L longitudinal axis of contact member
10 a tilt angle

1-13. (canceled)

14. A soft and dry electrode for measuring bioelectric signals of an individual, the electrode made of elastomeric material, has conductive properties, and comprises a central connector portion with attachment means for attaching the electrode to a measuring apparatus and a plurality of flexible contact members,
wherein the electrode has a connector side on a side of the attachment means, and a contact side opposite the connector side and facing the individual when applying the electrode to the individual;
wherein the connector portion further defines a central axis of the electrode arranged centrally through the contact side and the connector side of the electrode;
wherein the plurality of contact members is arranged around the central axis and connected with the connector portion, the plurality of contact members further protrudes on the contact side of the connector portion for contacting an area of interest of the individual for measurement;
wherein each contact member of the plurality of contact members comprises a flexible leg portion and a foot portion at a free end of the flexible leg portion;
wherein the flexible leg portion has a longitudinal cavity formed by an inner wall curved around a longitudinal axis of the contact member,
wherein the longitudinal cavity remains open on the connector side and is closed on the contact side by the foot portion; and
wherein the foot portion and the flexible leg portion form a continuous radially inner contact surface.

15. The electrode according to claim 14, wherein the radially inner contact surface is tilted, and a tilt angle between the radially inner contact surface and the central axis of the electrode is about 10 to about 45 degrees and opens on the contact side.

16. The electrode according to claim 15, wherein the tilt angle of the contact surface starting at a lower end of the leg portion to the lower end of the foot portion continuously increases.

17. The electrode according to claim 15, wherein the tilt angle of the contact surface starting at a lower end of the leg portion to the lower end of the foot portion continuously increases to about 35 to about 45 degrees.

18. The electrode according to claim 14, wherein a thickness of the curved inner wall decreases in a radially outward direction.

19. The electrode according to claim 14, wherein the foot portion extends in a radial outward direction over an inner wall of the leg portion.

20. The electrode according to claim 14, wherein the leg portion further comprises a curved outer wall forming together with the curved inner wall the longitudinal cavity of the leg portion with an opening towards the connector side of the electrode.

21. The electrode according to claim 20, wherein a thickness of the curved outer wall is smaller than a thickness of the curved inner wall.

22. The electrode according to claim 20, wherein an outer surface of the inner wall and/or an outer surface of the outer wall translates into the outer surface of the foot portion to form a continuous outer surface of the contact element.

23. The electrode according to claim 14, wherein each leg portion is connected to adjacent leg portions at an upper end of the leg portion.

24. The electrode according to claim 14, wherein the attachment means is a knob formed by the connector portion or a rigid connecting insert embedded in the connector portion of the electrode.

25. The electrode according to claim 14, wherein the longitudinal cavity has a semi-oval, semi-elliptic, oval or elliptic cross section.

26. The electrode according to claim 14, wherein the longitudinal cavity is open or closed in a radially outer direction.

27. The electrode according to claim 14, wherein the connector portion and the plurality of contact members are integrally formed as single pieces of elastomeric material, with or without a rigid insert in the connector portion.
